(19) United States
(12) Patent Application Publication
(10) Pub. No.: US 2004/0215291 A1
(43) Pub. Date: Oct. 28, 2004

(54) Programmer storage of implantable medical device settings

(76) Inventor: Maarten Van Bentem, Dieren (NL)

Correspondence Address: SHUMAKER & SIEFFERT, P.A., Suite 105, 8425 Seasons Parkway, St. paul, MN, 55125, US

(21) Appl. No.: 10/423,034

(22) Filed: Apr. 24, 2003

(51) Int. Cl. A61N 1/08

(52) U.S. Cl. 607/60

(57) ABSTRACT

The invention is directed to the storage of patient-specific settings in a programmer of an implantable medical device (IMD). Specifically, following the communication of patient-specific settings from the programmer to the IMD, the programmer can maintain the patient-specific settings in memory for later use. If the IMD is reset, the programmer can re-communicate the same patient-specific settings without requiring a technician to re-load the programmer with the original settings. Also, when a subsequent communication session is established between the IMD and the programmer, the programmer can display the stored settings without requiring an upload from the IMD, which can save time and power.

### FIELD OF THE INVENTION

The invention relates to implantable medical devices (IMDs), and more particularly to programmers that telemetrically communicate with IMDs.

### BACKGROUND OF THE INVENTION

A wide variety of implantable medical devices (IMDs) have been developed in order to monitor patient conditions and deliver therapy to the patient. An IMD typically includes a hermetically sealed housing coupled to one or more leads that are surgically implanted inside a patient for short or long term therapy. The IMD may provide therapeutic stimulation to the patient or deliver drugs or agents to the patient. Alternatively or additionally, the IMD may have sensing or monitoring capabilities. For example, the IMD may sense information within a patient and store the sensed information for subsequent analysis. Telemetry can be used to communicate sensed information from the IMD to a programmer so that analysis can be performed.

One common example of an IMD is a pacemaker. A pacemaker typically includes a hermetically sealed housing and one or more pacing and sensing leads coupled to the housing for delivery of pacing pulses to a patient's heart and sensing of heart conditions. Another example of an IMD is a combination pacemaker-cardioverter-defibrillator. Other examples include implantable brain stimulators, implantable gastric system stimulators, implantable nerve stimulators or muscle stimulators, implantable lower colon stimulators, implantable drug or beneficial agent dispensers or pumps, implantable cardiac signal loops or other types of recorders or monitors, implantable gene therapy delivery devices, implantable incontinence prevention or monitoring devices, implantable insulin pumps or monitoring devices, and so on.

Telemetry generally refers to communication of data, instructions, and the like between an IMD and a programmer. For example, the programmer can use telemetry to program patient-specific settings into a medical device in order to define the therapy to be delivered to the patient by the IMD. In addition, the programmer may use telemetry to interrogate the IMD in order to acquire diagnostic data, event marker data, activity data and other data collected or identified by the IMD. The acquired data may then be used to identify new or modified therapies that should be programmed into the IMD.

Telemetry typically involves wireless communication between the IMD and the programmer using radio frequency (RF) signals, infrared (IR) frequency signals, or other electromagnetic signals. Any of a variety of modulation techniques may be used to modulate data on a respective electromagnetic carrier wave. Alternatively, telemetry may be performed using transcutaneous wired connections, sound waves, or even the patient's flesh as the transmission medium. A number of different telemetry systems and techniques have been developed to facilitate the transfer of data between a medical device and the associated programmer.

Sometimes an IMD is reset following implantation in a patient. When this occurs, the patient-specific settings that were telemetrically programmed into the IMD can be lost. Thus, re-programming of the patient-specific settings of the IMD is commonly performed following a reset. In that case, a technician typically obtains the programmed settings from various paper work that was documented by the physician and documented during the production of the IMD, and then manually reloads the settings into the programmer. The programmer can then be used to re-program the original settings into the IMD.

### BRIEF SUMMARY OF THE INVENTION

In general, the invention is directed to the long-term storage of patient-specific settings in a programmer of an implantable medical device (IMD). Specifically, following the communication of patient-specific settings from the programmer to the IMD, the programmer can maintain the patient-specific settings in memory for later use. If the IMD is reset, the programmer can re-communicate the same patient-specific settings without requiring a technician to manually re-load the programmer with the original settings, and may also communicate non-patient specific parameters, e.g., IMD-parameters, to the IMD without requiring a technician to manually load the programmer with the parameters. Also, when a subsequent communication session is established between the IMD and the programmer, the programmer can display the stored settings without requiring an upload from the IMD, which can save time and power. The programmer may perform a checksum comparison on the settings to verify that the settings stored in the programmer are the same as the settings stored in the IMD. If so, the programmer can display the patient-specific settings stored in the programmer as being the patient-specific settings stored in the IMD.

In one embodiment, the invention provides a method comprising receiving, in a programmer, patient-specific settings for an implantable medical device, and communicating the patient-specific settings from the programmer to the implantable medical device. The method further comprises storing the patient-specific settings in the implantable medical device, and storing the patient-specific settings in the programmer for an extended period for use after the patient-specific settings have been stored in the implantable medical device.

In another embodiment, the invention provides a programmer for an implantable medical device comprising a control unit and a user interface coupled to the control unit to receive patient-specific settings for an implantable medical device. The programmer also includes a telemetry unit to communicate the patient-specific settings from the programmer to the implantable medical device, and a memory to store the patient-specific settings in the programmer for an extended period after the patient-specific settings have been communicated to the implantable medical device.

In another embodiment, the invention provides a system comprising an implantable medical device and a programmer that communicate via telemetry. In particular, the programmer receives patient-specific settings for the implantable medical device and communicates the patient-specific settings from the programmer to the implantable medical device. The implantable medical device stores the patient-specific settings in the implantable medical device, and the programmer stores the patient-specific settings in the programmer for an extended period for use after the patient-specific settings have been stored in the implantable medical device.

In another embodiment, the invention provides a programmer for an implantable medical device comprising means for loading patient-specific settings for an implantable medical device into a programmer, means for communicating the patient-specific settings from the programmer to the implantable medical device, and means for storing the patient-specific settings in the programmer for an extended period for use after the patient-specific settings have been stored in the implantable medical device.

### BRIEF DESCRIPTION OF THE DRAWINGS

FIG. 1 is a conceptual view of a programmer telemetrically communicating with a pacemaker according to an embodiment of the invention.

FIG. 2 is a block diagram of a system that includes an implantable medical device (IMD) that telemetrically communicates with a programmer according to an embodiment of the invention.

FIGS. 3-5 are flow diagrams illustrating techniques for storing and using patient-specific IMD settings in a programmer according to embodiments of the invention.

### DETAILED DESCRIPTION OF THE INVENTION

The invention is directed to long-term storage of patient-specific settings in a programmer of an implantable medical device (IMD). For example, following the communication of patient-specific settings from the programmer to the IMD, the programmer maintains the patient-specific settings in memory for an extended period. Then, if the IMD is reset, the programmer re-communicates the same patient-specific settings without requiring a technician to manually re-load the programmer with the original settings based on paper records. The invention exploits the fact that a high percentage of IMD patients generally return to the same hospital for follow-up and maintenance of the IMD. In accordance with the invention, the programmer that originally programmed the IMD maintains a copy of the settings for later use, if needed.

In addition, the invention can improve subsequent communication sessions between the IMD and the programmer that originally programmed the patient-specific settings into the IMD. For example, when a subsequent communication session is established between the IMD and the programmer, the programmer can display the stored patient-specific settings without requiring an upload from the IMD, which can save time and IMD battery power. The programmer may perform a checksum comparison on the patient-specific settings to verify that the settings stored in the programmer are the same as the settings stored in the IMD. If so, the programmer can display the patient-specific settings stored in the programmer as being the patient-specific settings stored in the IMD. In this manner, the physician or clinician can ensure that the settings are up-to-date and have not been lost or corrupted.

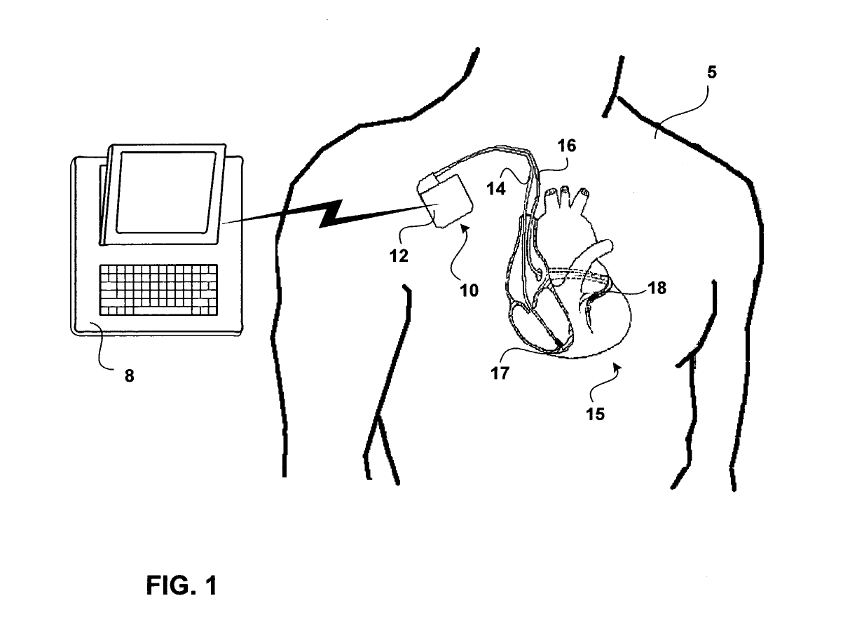

FIG. 1 is a simplified schematic view of pacemaker 10 within a patient 5. Pacemaker 10 represents one exemplary embodiment of an IMD according to the invention. Pacemaker 10 generally includes a hermetically sealed housing 12, and one or more pacing and sensing leads 14 and 16 coupled to housing 12. Leads 14, 16 each position one or more electrodes 17, 18 with respect to heart 15 of patient 5. Electrodes 17, 18 sense electrical signals attendant to the depolarization and repolarization of heart 15, and deliver pacing pulses generated by pacemaker device 10 for causing depolarization of cardiac tissue in the vicinity of the respective electrode 17, 18. Electrodes 17, 18 may comprise unipolar or bipolar electrodes, as are well known in the art. Any number of leads may be used.

Implantable leads 14, 16 may include any number of additional electrodes (not shown) distributed along the length of the respective lead. Electrodes 17, 18 or other electrodes may be used for sensing and/or delivery of stimulation pulses. Additional electrodes (not shown) may also be used for delivery of high voltage defibrillation or cardioversion shocks.

Programmer 8 telemetrically communicates with pacemaker 10 in order to program patient-specific settings into pacemaker 10 and readout IMD-specific parameters. In particular, programmer 8 can telemetrically communicate patient-specific information that defines one or more pacing modes, pacing pulse widths, pacing pulse amplitudes, blanking periods, pace conditioning algorithms, sensing thresholds or the like. Also, programmer 8 can telemetrically readout non-patient specific IMD information that defines calibration information specific to pacemaker 10, which was obtained during the production of pacemaker 10 and stored into the internal memory of pacemaker 10. Typically, a physician or clinician selects the patient-specific settings and inputs then to programmer 8 for communication to pacemaker 10. In this manner, pacemaker 10 can be customized to deliver patient-specific therapy. Programmer 8 may, for example, communicate with pacemaker 10 by wireless transmission via a programming head (not shown) placed over pacemaker 10, e.g., on the skin of patient 5.

In accordance with the invention, programmer 8 maintains for an extended period a copy of the patient-specific settings communicated to pacemaker 10. In other words, following the communication of patient-specific settings from programmer 8 to pacemaker 10 and the storage of these settings in pacemaker 10, programmer 8 archives the settings in a long-term memory, e.g., non-volatile memory such as a hard drive, or the like. If pacemaker 10 is reset or somehow loses its patient-specific settings, programmer 8 can access and then reload the settings into pacemaker 10 without the need for a technician to manually identify the settings and re-load programmer 8. In order to exploit the settings stored in programmer 8 following a reset of pacemaker 10, patient 5 may return to the same hospital where programming of pacemaker 10 was performed. In that case, programmer 8 can be located or identified at that hospital, and then used to re-load the settings into pacemaker 10.

Also, by storing the patient-specific settings in programmer 8, the invention can improve subsequent communication sessions between pacemaker 10 and programmer 8 even if a re-load of the settings is not needed. For example, when a subsequent communication session is established between pacemaker 10 and programmer 8, programmer 8 can display the stored settings to a physician without requiring an upload of the settings from pacemaker 10, which can save time and reduce power consumption by pacemaker 10. The reduced power consumption helps to conserve the limited battery resources within pacemaker 10. Instead, programmer 8 can simply identify pacemaker 10, access the stored settings for pacemaker 10, and display the settings stored in programmer 10 to the physician as being the settings of pacemaker 10. In some cases, programmer 10 may perform a checksum comparison on the settings to verify that the settings stored in programmer 8 are indeed the same as the settings stored in pacemaker 10.

A checksum refers to an error detection scheme in which a value is assigned to the patient-specific settings based on the number of bits in the settings. Thus, the value assigned to the settings by the checksum provides a reference that can be compared to another value associated with other settings, e.g., stored in pacemaker 10 and communicated from pacemaker 10 to programmer 8. If the values match, then the settings should also match. In other words, programmer 8 performs a checksum comparison by generating a value based on the patient-specific settings stored by programmer 8 and comparing the generated value to a similar value generated by pacemaker 10 based on its stored settings. If the values match, the settings in programmer 8 likely match those stored in pacemaker 10. In this manner, the checksum comparison can be performed on the patient-specific settings to verify that the settings stored in programmer 8 are indeed the same as the settings stored in pacemaker 10. Again, communication of a checksum value from pacemaker 10 to programmer 8 may use significantly less pacemaker battery power than communication of the settings.

Programmer 8 may be used to program a plurality of pacemakers similar to pacemaker 10. Accordingly, programmer 8 can store the settings for the different pacemakers. When another communication session is established between programmer 8 and one of the pacemakers, programmer 8 simply identifies the given pacemaker, e.g., via a signature, and accesses the stored settings for that pacemaker. Such techniques can facilitate a re-load of the settings without the need to reprogram the settings into programmer 8, or can facilitate the display of the settings without requiring an upload of the settings from the given pacemaker.

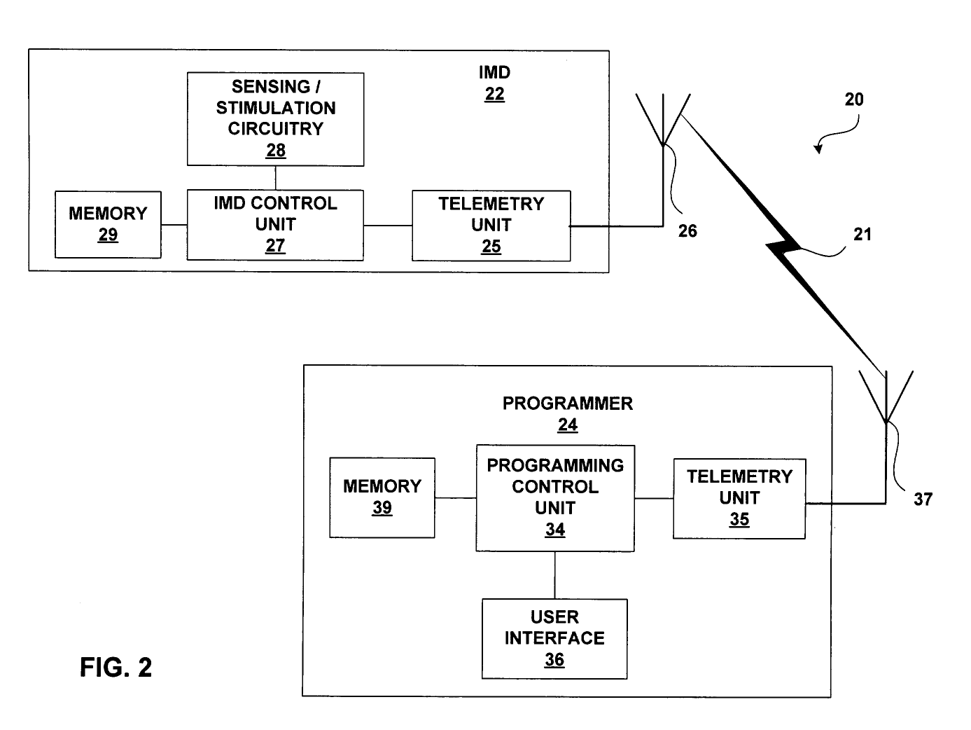

FIG. 2 is a block diagram of a system 20 comprising an IMD 22 and a programmer 24. IMD 22 may correspond to pacemaker 10 (FIG. 1), and programmer 24 may correspond to programmer 8. Alternatively, IMD 22 can comprise any of a wide variety of other IMDs, with programmer 24 being the corresponding programmer for the given IMD. In system 20, programmer 24 and IMD 22 communicate via telemetry signals 21. Any of a wide variety of telemetry techniques may be used to facilitate transfer of information between IMD 22 and programmer 24.

IMD 22 includes a telemetry unit 25 and an antenna 26 which facilitate transmission and reception of telemetry signals 21 to and from programmer 24. IMD 22 also includes sensing and stimulation circuitry 28 for sensing and/or stimulating a patient for therapeutic purposes. For example, sensing/stimulation circuitry 28 may include electrodes disposed on medical leads and implanted at locations in a patient where sensing and stimulation occurs. Sensing/stimulation circuitry 28 typically includes one or more amplifiers to enhance the sensed signals and to generate the electrical potentials needed for effective stimulation.

IMD control unit 27 coordinates circuitry 28 so that sensing and stimulation occurs at proper times. In particular, IMD control unit 27 can execute various sensing and stimulation algorithms that define the therapy to be provided. For example, if IMD 22 is a cardiac pacemaker, IMD control unit 28 may execute algorithms that receive sensed information from circuitry 28 and determine proper times for delivery of pacing pulses. Also, if IMD control unit 27 identifies an arrhythmia, it may respond by causing circuitry 28 to provide stimulation therapy responsive to the identified arrhythmia. IMD control unit 27 can execute a number of algorithms to identify and respond to a wide variety of potential arrhythmias in the patient's heart.

IMD 22 also includes memory 29 coupled to IMD control unit 27. Memory 29 may comprise random access memory (RAM), read-only memory (ROM), non-volatile random access memory (NVRAM), electrically erasable programmable read-only memory (EEPROM), flash memory, various combinations, or the like. Memory 29 can store various pacing algorithms executed by IMD control unit 27. Also, memory 29 may store patient-specific settings and possibly IMD-specific parameters.

The patient-specific settings can be loaded into IMD 22 via telemetry signals 21 sent from programmer 24, and may define such things as pacing modes, pacing pulse widths, pacing pulse amplitudes, blanking periods, pace conditioning algorithms, sensing thresholds or the like. IMD-specific parameters may be programmed into IMD 22 during manufacture, and may include the algorithms executed by IMD control unit 27, voltage references, offset values for amplifiers of circuitry 28, calibration values for an accelerometer, measured capacitance values of capacitors, or the like. If desired, memory 29 may also be used to store diagnostic information sensed by circuitry 28, e.g., for communication to programmer 22 so a physician can better evaluate and diagnose the patient.

Programmer 24 also includes a telemetry unit 35 and an antenna 37 which facilitate transmission and reception of telemetry signals 21 to and from IMD 22. Programming control unit 34 coordinates operation of programmer 24. User interface 36, such as a touch screen display, keypad, or the like, allows a user to input algorithms, settings, or other parameters, into programmer 24 for communication to IMD 22. The user, for example, is typically a physician or clinician that uses programmer 24 to program operation of IMD 22 via telemetry.

Programming control unit 34 receives information input by the user via user interface 36, and communicates the information to IMD 22 via telemetry unit 35 and antenna 37. For example, the user can load patient-specific settings into programmer 24, via user interface 36, for communication to IMD 22. The patient-specific settings may define such things as pacing modes, pacing pulse widths, pacing pulse amplitudes, blanking periods, pace conditioning algorithms, sensing thresholds or the like. Once received by IMD 22, the patient-specific settings can be stored in memory 29 to define patient-specific operation of IMD 22 in accordance with the settings.

Programmer 24 also includes a memory 39, such as random access memory (RAM), read-only memory (ROM), non-volatile random access memory (NVRAM), electrically erasable programmable read-only memory (EEPROM), flash memory, hard-disk, floppy disk, magnetic tape, CD-R disk, CD-RW disk, combinations of different memories, or the like. In accordance with the invention, memory 39 of programmer 24 maintains for an extended period, a copy of the patient-specific settings loaded into IMD 22. Therefore, if such settings need to be reloaded into IMD 22, e.g., following a reset of IMD 22, programmer 24 can quickly access the settings form memory 39 to facilitate the reload without the need for a clinician or physician to obtain the settings from paper records and reload the settings into programmer via user interface 36. In order to support such long-term storage in a robust and reliable manner, memory 39 typically includes at least some non-volatile memory.

By storing the patient-specific settings in memory 39 programmer 24, the invention can also improve subsequent communication sessions between IMD 22 and programmer 24 even if a re-load of the settings is not needed. For example, when a subsequent communication session is established between IMD 22 and programmer 24, programmer 24 can display the stored patient-specific settings associated with IMD 22 via user interface 36. In other words, because the patient-specific settings are stored in memory 39 of programmer 24, programmer 24 does not need to upload the settings from IMD 22 in order to display the settings to a user. Instead, programmer 24 can simply identify IMD 22, access the stored settings from memory 39 for IMD 22, and display the patient-specific settings to the physician via user interface 36 as being the settings of IMD 22.

In some cases, programming control unit 34 performs a checksum comparison on the settings to verify that the patient-specific settings stored in programmer 24 are the same as the patient-specific settings stored in IMD 22. In that case, rather than communicate its patient-specific settings to programmer 24, IMD 22 may simply identify itself, e.g., by communicating a signature, and can communicate a checksum value instead of communicating the settings. Programmer 24 can access the patient-specific settings for IMD 22 in from memory 39, generate another checksum value based on its stored settings and compare its generated checksum value to that communicated from IMD 22 to verify that the patient-specific settings stored in programmer 24 are the same as the patient-specific settings stored in IMD 22. If so, programmer 24 displays the patient-specific settings stored in memory 39 for the given IMD, as being the settings stored in that IMD. In this manner, storage of the patient-specific settings of IMD 22 in memory 39 of programmer 24 can simplify subsequent communication sessions between IMD 22 and programmer 24.

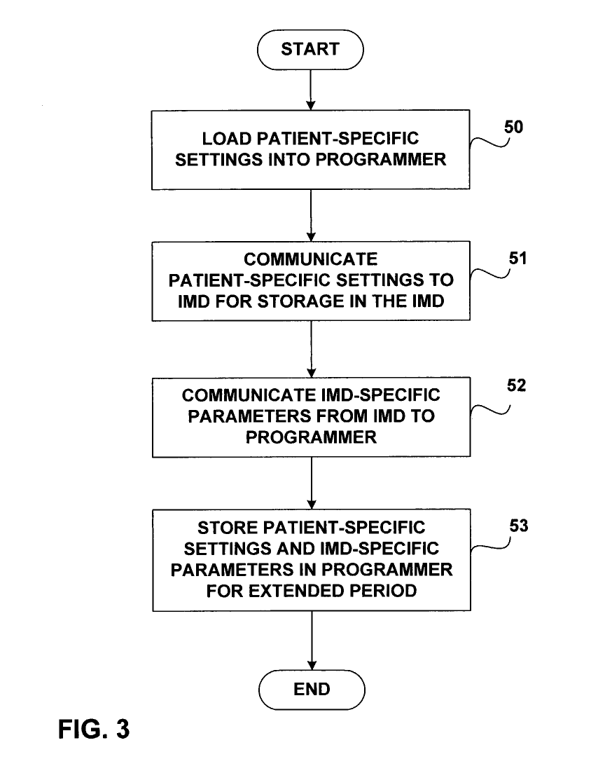

FIG. 3 is a flow diagram illustrating a technique for storing patient-specific IMD settings in a programmer according to an embodiment of the invention. As shown in FIG. 3, a user loads patient-specific settings into an IMD programmer 24 (50). For example, the user is typically a physician, clinician, technician, or the like, and the patient-specific settings typically comprise modes of operation, pulse widths or pulse amplitudes, blanking periods, pace conditioning algorithms or sensing thresholds, although the invention is not limited in these respects. Once programmer 24 has received the patient-specific settings, it communicates the patient-specific settings to the appropriate IMD 22 (51), where the settings are stored. In addition, IMD 22 communicates its IMD-specific parameters back to programmer 24 for storage in programmer 24 (52). In accordance with the invention, the patient-specific settings and IMD-specific parameters are stored in programmer 24 for an extended period (53), e.g., for later use.

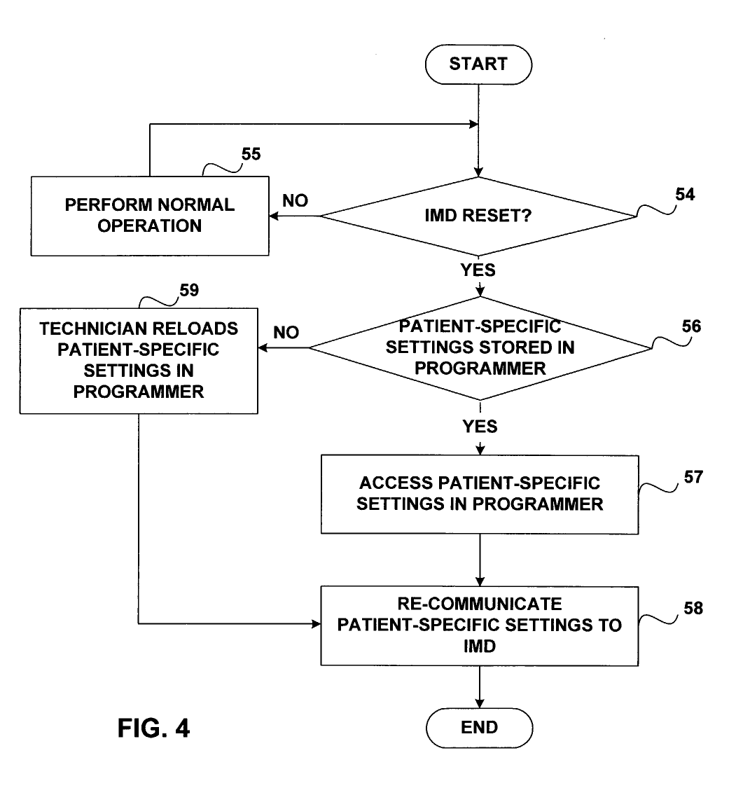

FIG. 4 is another flow diagram illustrating a technique for using the patient-specific settings stored in programmer 24. As shown in FIG. 4, IMD 22 generally performs its normal operations (55) until it is reset (yes branch of 54). If IMD 22 is reset (yes branch of 54), programmer 24 identifies whether the patient-specific settings for that IMD are stored in programmer 24 (56), e.g., by comparing a communicated IMD signature to stored signatures associated with the stored settings in programmer 24. If programmer 24 has stored the patient-specific settings for IMD 22 (yes branch of 56), programmer 24 accesses the stored settings for that IMD (57). Programmer 24 then re-communicates the patient-specific settings to the IMD (58). For example, programmer 24 can access the patient-specific settings for that IMD based on the received signature, and can re-communicate the patient-specific settings to the IMD. In this manner, the need for a technician to identify the settings, e.g., from paperwork, and re-load the settings into programmer 24 can be avoided.

As further shown in FIG. 4, if programmer 24 has not stored the patient-specific settings for IMD 22 (no branch of 56), then a technician reloads the patient-specific settings for IMD 22 into programmer 24 (59). In that case, a technician typically obtains the programmed settings from various paper work that was documented by the physician and then manually reloads the settings into programmer 24. Programmer 24 then re-communicates the patient-specific settings to the IMD (56). In accordance with the invention, the need for a technician to manually reload the patient-specific settings into programmer 24 can be avoided when the patient specific settings are stored in programmer 24 for an extended period.

In most cases, programmer 24 stores the patient-specific settings for every IMD that it programs. The patient-specific settings for each IMD can be archived based on a signature associated with each IMD, such as a unique identifying number for the given IMD. Thereafter, if one of the IMDs needs re-programming, programmer 24 can access the proper settings based on the signature of the given IMD, and can re-communicate the settings to that IMD.

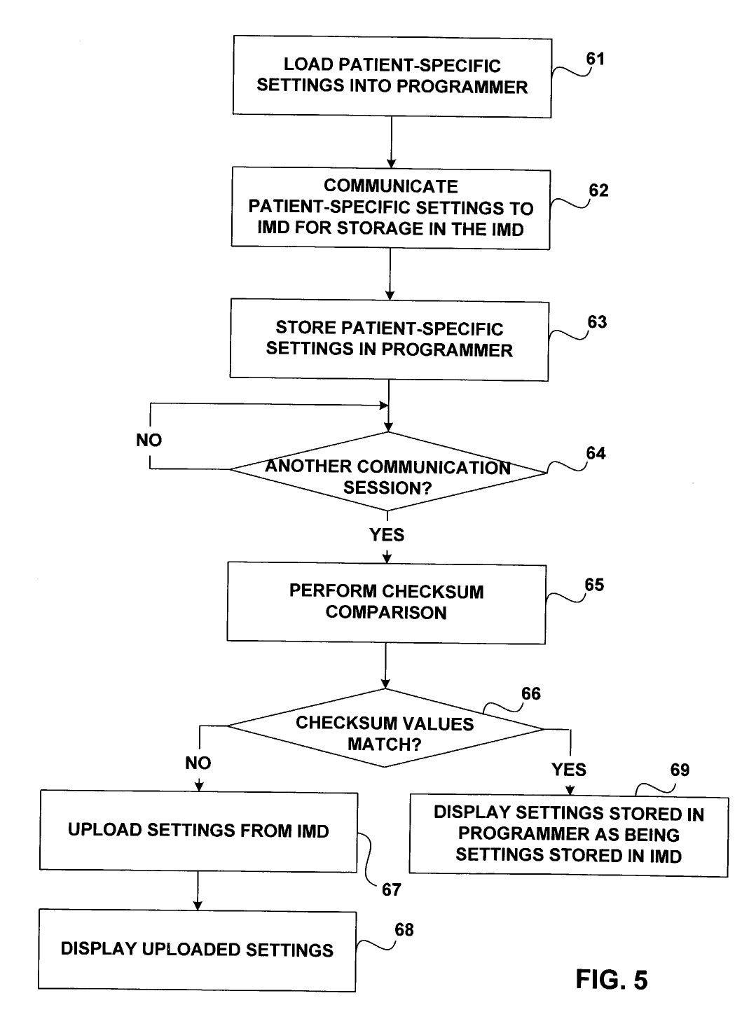

FIG. 5 is another flow diagram illustrating a technique for using patient-specific IMD settings stored in a programmer for an extended period according to an embodiment of the invention. As shown in FIG. 5, a user loads patient-specific settings into an IMD programmer 24 (61), and programmer 24 communicates the received patient-specific settings to an IMD 22 (62), which stores the settings. In addition, the patient-specific settings are also stored in programmer 24 (63) for later use.

Thereafter, if another communication session is established between programmer 24 and the same IMD 22 (yes branch of 64), programmer 24 makes use of the stored settings associated with IMD 22 even if a long period is between these sessions. For example, rather than upload the patient-specific settings from IMD 22, programmer 24 may perform a checksum comparison (65) to identify whether the settings stored in programmer 24 are the same as those in IMD 22. Specifically, IMD 22 generates a checksum value based on its stored settings and communicates this checksum value to programmer 24 along with a signature that identifies IMD 22. Programmer 24 similarly generates a checksum value based on the patient-specific settings stored in programmer for IMD 22, which can be accessed based on the received signature.

If the checksum values match (yes branch of 66), programmer 24 displays the patient-specific settings stored in programmer 24 as being the patient-specific settings of IMD 22 (69). In that case, programmer 24 does not request IMD 22 to send its patient-specific settings. In other words, in that case, an upload of the settings from IMD 22 to programmer 24 can be avoided, saving time and IMD battery power. However, if the checksum values do not match (no branch of 66) the upload from IMD 22 is performed (67), and programmer 24 displays the uploaded settings (68). In other words, if the checksum values do not match (no branch of 66), programmer 24 requests IMD 22 to send its patient-specific settings to programmer 24 for display to the user.

Again, in most cases, programmer 24 stores the patient-specific settings for every IMD that it programs. The patient-specific settings for each IMD can be archived based on a signature associated with each IMD, such as a unique identifying number. Techniques described herein may include communicating first patient-specific settings from programmer 24 to a first implantable medical device, storing the first patient-specific settings in the implantable medical device, and storing the first patient-specific settings in programmer 24 for use after the first patient-specific settings have been stored in the first implantable medical device. Moreover, the techniques may further include communicating second patient-specific settings from programmer 24 to a second implantable medical device, storing the second patient-specific settings in the second implantable medical device, and storing the second patient-specific settings in programmer 24 for use after the second patient-specific settings have been stored in the second implantable medical device.

Thereafter, if a first IMD establishes a communication session with programmer 24, programmer 24 accesses first patient-specific settings from memory 39 for the first IMD. Similarly, if a second IMD establishes a communication session with programmer 24, programmer 24 accesses second patient-specific settings from memory 39 for the second IMD, and so forth.

Also, in some cases, it may be desirable to identify, communicate and possibly display not only the patient-specific settings of the given IMD, but also IMD-specific parameters such as algorithms executed by IMD control unit 27. Then, IMD-specific parameters can be sent back to the IMD following a reset. Again, other exemplary IMD-specific parameters typically include voltage references, offset values for various amplifiers of circuitry 28, calibration values for an accelerometer, measured capacitance values of capacitors, or the like. If programmer 24 also stores such IMD-specific parameters, a checksum comparison technique can also be applied to the IMD-specific parameters relative to the IMD-specific parameters stored in IMD 24. If the checksum values match for the IMD-specific parameters, programmer 22 can avoid an upload of the IMD-specific parameters during a subsequent communication session.

A number of embodiments of the invention have been described. However, one skilled in the art will appreciate that the invention can be practiced with embodiments other than those disclosed. For example, although many details of the invention have been provide in the context of a cardiac pacemaker, the same techniques may also be applied with any IMD and associated programmer. In addition, the patient-specific settings and IMD-specific parameters listed herein are exemplary. The invention can be practiced with a wide variety of other patient-specific settings or IMD-specific parameters. Also, in some systems the patient-specific settings listed herein may be IMD-specific parameters, and vice versa. For example, specific pacing algorithms may be patient-specific or non-patient-specific, depending on the implementation.

The various components of IMD 22 and programmer 24 may be implemented within an application specific integrated circuit (ASIC), a field programmable gate array (FPGA), a programmable logic device, specifically designed hardware components, one or more processors, or any combination thereof. If implemented in software, various components of IMD 22 and programmer 24 may be embodied in a computer readable medium that stores computer readable instructions, e.g., program code, that can be executed by a processor, DSP or other logic device to carry out one of more of the techniques described above. For example, the computer readable medium may comprise memory 29 or 39, such as random access memory (RAM), read-only memory (ROM), non-volatile random access memory (NVRAM), electrically erasable programmable read-only memory (EEPROM), flash memory, or the like. The computer readable medium may comprise computer readable instructions that when executed in an IMD to carry out one or more of the techniques described herein. The disclosed embodiments are presented for purposes of illustration and not limitation, and the invention is limited only by the claims that follow.

What is claimed is:

1. A programmer for an implantable medical device (IMD) comprising:
a control unit;
a user interface coupled to the control unit to receive patient-specific settings for an implantable medical device;
a telemetry unit to communicate the patient-specific settings from the programmer to the implantable medical device; and
a memory that stores the patient-specific settings in the programmer for an extended period after the patient-specific settings have been communicated to the implantable medical device.

2. The programmer of claim 1, wherein the telemetry unit re-communicates the patient-specific settings to the implantable medical device following a reset of the implantable medical device.

3. The programmer of claim 1, wherein the telemetry unit receives IMD-specific parameters and the memory stores the IMD-specific parameters for the extended period.

4. The programmer of claim 1, wherein the control unit accesses the patient-specific settings from the memory in response to the programmer establishing a communication session with the implantable medical device.

5. The programmer of claim 4, wherein during the communication session the control unit performs a checksum comparison to ensure that the patient-specific settings in the programmer are the same as the patient-specific settings in the implantable medical device.

6. The programmer of claim 5, wherein during the communication session the control unit performs the checksum comparison to further ensure that IMD-specific parameters stored in the programmer are the same as IMD-specific parameters stored in the implantable medical device.

7. The programmer of claim 5, wherein during the communication session the control unit:
identifies from the checksum comparison that the patient-specific settings in the programmer are not the same as the patient-specific settings in the implantable medical device; and
requests the implantable medical device to communicate the patient-specific settings in the implantable medical device to the programmer.

8. The programmer of claim 5, wherein during the communication session the control unit:
identifies from the checksum comparison that the patient-specific settings in the programmer are the same as the patient-specific settings in the implantable medical device; and
does not request the implantable medical device to communicate the patient-specific settings in the implantable medical device to the programmer.

9. The programmer of claim 8, wherein the user interface includes a display that displays the patient-specific settings stored in the programmer as being the patient-specific settings stored in the implantable medical device.

10. The programmer of claim 5, wherein during the communication session the programmer receives from the implantable medical device a checksum value for use in the checksum comparison.

11. A method comprising:
receiving in a programmer, patient-specific settings for an implantable medical device (IMD);
communicating the patient-specific settings from the programmer to the implantable medical device;
storing the patient-specific settings in the implantable medical device; and
storing the patient-specific settings in the programmer for an extended period for use after the patient-specific settings have been stored in the implantable medical device.

12. The method of claim 11, further comprising re-communicating the patient-specific settings from the programmer to the implantable medical device following a reset of the implantable medical device.

13. The method of claim 11, further comprising following storing the patient-specific settings in the implantable medical device:
establishing a communication session between the implantable medical device and the programmer; and
accessing the patient-specific settings in the programmer in response to establishing the communication session.

14. The method of claim 13, further comprising performing a checksum comparison to ensure that the patient-specific settings in the programmer are the same as the patient-specific settings in the implantable medical device.

15. The method of claim 14, further comprising performing the checksum comparison to further ensure that IMD-specific parameters stored in the programmer are the same as IMD-specific parameters stored in the implantable medical device.

16. The method of claim 14, further comprising:
identifying from the checksum comparison that the patient-specific settings in the programmer are not the same as the patient-specific settings in the implantable medical device; and
communicating the patient-specific settings in the implantable medical device to the programmer.

17. The method of claim 14, further comprising:
identifying from the checksum comparison that the patient-specific settings in the programmer are the same as the patient-specific settings in the implantable medical device; and
not communicating the patient-specific settings in the implantable medical device to the programmer.

18. The method of claim 17, further comprising displaying the patient-specific settings stored in the programmer as being the patient-specific settings stored in the implantable medical device.

19. The method of claim 15, further comprising:
identifying from the checksum comparison that the IMD-specific parameters in the programmer are not the same as the IMD-specific parameters in the implantable medical device; and
communicating the IMD-specific parameters in the implantable medical device to the programmer.

20. The method of claim 11, further comprising
communicating first patient-specific settings from the programmer to a first implantable medical device;
storing the first patient-specific settings in the implantable medical device;
storing the first patient-specific settings in the programmer for use after the first patient-specific settings have been stored in the first implantable medical device;
communicating second patient-specific settings from the programmer to a second implantable medical device;
storing the second patient-specific settings in the second implantable medical device; and
storing the second patient-specific settings in the programmer for use after the second patient-specific settings have been stored in the second implantable medical device.

21. The method of claim 20, further comprising following storing the first and second patient-specific settings in the implantable medical device:
establishing a communication session between the first implantable medical device and the programmer;
accessing the first patient-specific settings in the programmer in response to establishing the communication session between the first implantable medical device and the programmer
establishing a communication session between the second implantable medical device and the programmer; and
accessing the second patient-specific settings in the programmer in response to establishing the communication session between the second implantable medical device and the programmer.

22. A system comprising an implantable medical device and a programmer that communicate via telemetry, wherein:
the programmer receives patient-specific settings for the implantable medical device;
the programmer communicates the patient-specific settings from the programmer to the implantable medical device;
the implantable medical device stores the patient-specific settings in the implantable medical device; and
the programmer stores the patient-specific settings in the programmer for an extended period use after the patient-specific settings have been stored in the implantable medical device.

23. The system of claim 22, wherein the programmer re-communicates the patient-specific settings from the programmer to the implantable medical device following a reset of the implantable medical device.

24. The system of claim 22, wherein following storing the patient-specific settings in the implantable medical device:
the programmer establishes a communication session between the implantable medical device and the programmer; and
the programmer accesses the patient-specific settings in the programmer in response to establishing the communication session.

25. The system of claim 24, wherein in response to establishing the communication session the programmer performs a checksum comparison to ensure that the patient-specific settings in the programmer are the same as the patient-specific settings in the implantable medical device.

26. The system of claim 25, wherein:
the programmer identifies from the checksum comparison that the patient-specific settings in the programmer are the same as the patient-specific settings in the implantable medical device;
the implantable medical device does not communicate the patient-specific settings in the implantable medical device to the programmer; and
the programmer displays the patient-specific settings stored in the programmer as being the patient-specific settings stored in the implantable medical device.

27. A programmer for an implantable medical device comprising:
means for receiving patient-specific settings for an implantable medical device;
means for communicating the patient-specific settings from the programmer to the implantable medical device; and
means for storing the patient-specific settings in the programmer for an extended period for use after the patient-specific settings have been stored in the implantable medical device.

28. The programmer of claim 27, further comprising means for re-communicating the patient-specific settings from the programmer to the implantable medical device following a reset of the implantable medical device.

29. The programmer of claim 27, further comprising:
means for establishing a communication session between the implantable medical device and the programmer following communication of the patient-specific settings to the implantable medical device; and
means for accessing the patient-specific settings in the programmer in response to establishing the communication session.

30. The programmer of claim 29, further comprising means for performing a checksum comparison to ensure that the patient-specific settings in the programmer are the same as the patient-specific settings in the implantable medical device.

31. The programmer of claim 30, further comprising:
means for identifying from the checksum comparison that the patient-specific settings in the programmer are the same as the patient-specific settings in the implantable medical device; and
means for displaying the patient-specific settings stored in the programmer as being the patient-specific settings stored in the implantable medical device.
